(19) United States
(12) Patent Application Publication
(10) Pub. No.: US 2019/0261948 A1
(43) Pub. Date: Aug. 29, 2019

(54) SYSTEM AND METHOD FOR ULTRAFAST SYNTHETIC TRANSMIT APERTURE ULTRASOUND IMAGING

(71) Applicant: Mayo Foundation for Medical Education and Research, Rochester, MN (US)

(72) Inventors: Ping Gong, Rochester, MN (US); Pengfei Song, Rochester, MN (US); Shigao Chen, Rochester, MN (US)

(73) Assignee: Mayo Foundation for Medical Education and Research, Rochester, MN (US)

(21) Appl. No.: 16/333,861

(22) PCT Filed: Sep. 14, 2017
(86) PCT No.: PCT/US17/51496

§ 371 Date: Mar. 15, 2019

Related U.S. Application Data
(60) Provisional application No. 62/395,442, filed on Sep. 16, 2016.

(51) Int. Cl. A61B 8/00; A61B 8/14; G01S 7/52; G01S 15/89

(52) U.S. Cl. CPC A61B 8/4488; A61B 8/145; A61B 8/54; G01S 7/5202; G01S 15/8997; G01S 7/52047; G01S 15/8927; G01S 15/8959; G01S 7/52042

(57) ABSTRACT

Systems and methods for ultrafast synthetic transmit aperture (“USTA”) ultrasound imaging using coded virtual sources are described. The methods can implement spatially distinct or spatially overlapping sub-apertures with appropriate timing of transmission between the spatially overlapping sub -apertures. The systems and methods described here are capable of increasing signal-to-noise ratio (“SNR”) and spatial resolution to reduce frame rate.

### CROSS-REFERENCE TO RELATED APPLICATIONS

This application claims the benefit of U.S. Provisional Patent Application Ser. No. 62/395,442, filed on Sep. 16, 2016, and entitled “SYSTEM AND METHOD FOR ULTRAFAST SYNTHETIC TRANSMIT APERTURE ULTRASOUND IMAGING.”

### BACKGROUND

The development of ultrafast ultrasound imaging techniques offers great opportunities to new imaging technologies, such as shear wave elastography, ultrafast Doppler imaging, and diverging wave compounding. In compounded ultrafast imaging, high frame rate B-mode images are acquired by coherently combining several plane (or diverging) wave emissions with different tilted angles. The frame rate is significantly improved compared to conventional line-by-line focused B-mode imaging; however, the high frame rate is usually achieved by sacrificing other quality metrics such as image signal-to-noise ratio (“SNR”) and spatial resolution.

Many efforts have been made to develop optimal transmission sequences to break the trade-off among image SNR, spatial resolution, and frame rate in ultrafast imaging. A recently proposed technique, multiplane wave (“MW”) imaging investigates the SNR improvement in ultrafast imaging. Multiple plane waves with different tilted angles are encoded by a Hadamard matrix and emitted successively with very small interleaved time gaps (e.g., a few microseconds) during one transmission event (i.e. pulse-echo event). Then the received signals from different transmission events can be decoded to recover each of the titled plane waves to perform coherent compounding. This technique increases SNR in ultrafast imaging without sacrificing resolution or frame rate.

Synthetic transmit aperture (“STA”) imaging has been used to enhance image resolution due to its optimal focusing in both transmit and receive. Some methods convert signals obtained from plane wave imaging to STA data through either compressed sensing or delay-decoding in the frequency domain. One of the challenges with STA is to increase its frame rate and SNR.

Various transmission schemes to enhance SNR for STA imaging have been proposed. One of these approaches involved multiple elements transmission instead of single element excitation by creating a series of virtual sources, each of which is formed by a sub-aperture of multiple transducer elements. Another category is applying coding matrices (e.g., Hadamard matrix, S-sequence, delay encoded sequence) to spatially encode the entire transducer array. Each transmit element is assigned a coding factor to modify the transmitted pulse during each transmission event. The inverse of the coding matrix is then multiplied to the received RF signals to decode the signals either in the time or frequency domain. Then the decoded RF signals can be reconstructed following standard STA manner. The transmission power can thus be significantly increased over that of standard STA imaging with single element transmission. Temporal encoding can also be implemented by transmitting a longer coded pulse (e.g., chirp and Golay coding) to increase the ultrasound energy for each pulse echo event. These methods can be combined for the spatiotemporal encoding to further improve SNR.

### SUMMARY OF THE DISCLOSURE

The present disclosure provides a method for ultrafast synthetic transmit aperture (“USTA”) imaging with an ultrasound system. A series of virtual sources that define sub-apertures of ultrasound transducer elements in an ultrasound transducer array are selected by a computer system. At least some of these sub-apertures are spatially overlapping. Coded virtual sources are then generated by applying a coding matrix to the series of virtual sources with the computer system. Entries in the coding matrix define a characteristic (e.g., an amplitude, a phase, a polarity) of transmit signals to be applied to the sub-apertures in each of a plurality of different transmission events. Coded signal data are then acquired from a subject by transmitting ultrasound beams to the subject in each of the plurality of a single transmission events with the sub-apertures in the ultrasound system using the respective coded virtual sources and receiving coded echo signals in response thereto. The transmission of ultrasound beams using those sub-apertures that are spatially overlapping is spaced apart in time by a time interval. The coded signal data are decoded with the computer system using an inverse of the coding matrix, and an image of the subject is produced from the decoded signal data using the computer system.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a flowchart setting forth the steps of an example method for designing an imaging sequence using coded virtual sources.

FIG. 2 is an illustration of an example virtual source.

FIG. 3 depicts an example of transmitting ultrasound beams via sub-apertures according to coded virtual sources.

FIG. 4 is an example of ultrasound wave fronts generated using coded virtual sources in two different transmission events.

FIG. 5 depicts an example of transmitting ultrasound beams via spatially overlapping sub-apertures according to time-shifted coded virtual sources.

FIGS. 6A-6C depict examples of different USTA imaging sequences.

FIG. 7 is a flowchart setting forth the steps of an example method for USTA imaging.

FIG. 8 is a block diagram of an example ultrasound system that can implement the USTA methods described in the present disclosure.

### DETAILED DESCRIPTION

Described here are systems and methods for ultrafast synthetic transmit aperture (“USTA”) ultrasound imaging using virtual sources with overlapping sub-apertures. The systems and methods described here are capable of increasing signal-to-noise ratio (“SNR”) and spatial resolution without needing to reduce frame rate.

The USTA techniques described here generally include the following steps. A series of virtual sources with overlapping sub-apertures is created. A coding matrix, such as a Hadamard coding matrix, is then applied to the virtual sources. Short time intervals (e.g., intervals on the order of a few microseconds) are added between the emissions of virtual sources to allow for the spatial overlap of sub-apertures during a single transmission event. In preferred embodiments, the methods described here can be implemented using an ultrasound system; however, in other embodiments the USTA techniques can be applied to other acoustic imaging and measurement applications, including those using SONAR systems, RADAR systems, and seismic survey systems.

In the techniques described here, transducer elements shared by two or more sub-apertures emit multiple pulses, thereby increasing the energy and SNR of the imaging method. Consequently, the methods described here can provide a significant improvement to SNR compared to previous synthetic transmit aperture (“STA”) and diverging wave compounding imaging techniques, while also maintaining good spatial resolution without needing to lower frame rate. Both the SNR and spatial resolution enhancement can be adjusted by changing the f-number of the virtual sources, the number of virtual sources, the location of the virtual sources, and the number of transducer elements in each sub-aperture, thereby allowing flexible customization and optimization for different imaging applications.

The design of a given USTA transmission sequence includes selection of an f-number (fₙ) for the virtual sources; the lateral location (lₓ), axial location (l_{z}), or both, of each virtual source; and the time interval added between different virtual source emissions (Δt). These factors (fₙ, lₓ, l_{z}, Δt) can be flexibly adjusted to optimize the USTA transmission sequence for different imaging requirements (e.g., spatial resolution driven, SNR driven, frame rate driven). The creation of an example UTSA imaging sequence is described below.

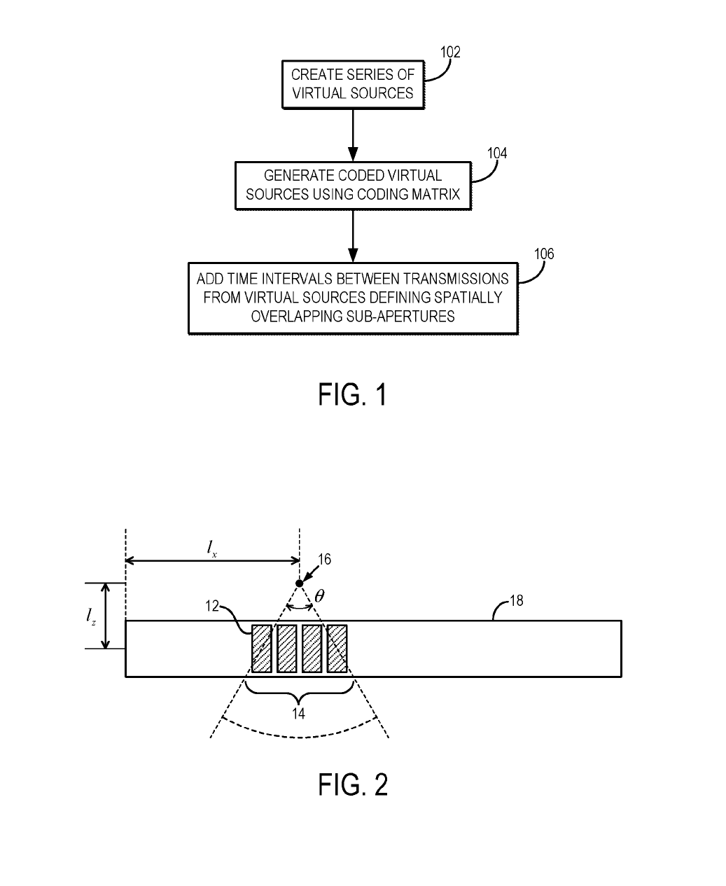

Referring now to FIG. 1, a flowchart is illustrated as setting forth an example of a method for generating a UTSA imaging sequence. The method includes creating a series of virtual sources, as indicated at step 102. A number of transmit elements (Nₑ) are used to create a virtual point source, as shown in FIG. 2. The transmit elements 12 associated with a virtual point define a sub-aperture 14. Appropriate time delays are applied to each element inside the sub-aperture 14 according to the lateral and axial coordinates (lₓ, l_{z}) of the virtual source 16. The virtual source 16 can be located either in front of or behind the transducer array 18 with a positive or negative f-number (fₙ), respectively, which can be calculated as,

$${f}_{n} = \frac{{I}_{z}}{{N}_{e} \cdot pitch} = \frac{1}{2 \cdot tan ( \frac{θ}{2} )} ; \qquad ( 1 )$$

where θ is the open angle of the virtual source. The size of the sub-aperture 14 defined by the virtual source 16 is Nₑ·pitch, which defines the open angle, θ, of the virtual source 16. In FIG. 2, a negative virtual source 16 results in the transmit elements 12 in the sub-aperture 14 emitting a de-focused, diverging ultrasound beam with transmitting power improved by approximately Nₑ times compared to single element firing. This increased transmitting power results in an \sqrt{Nₑ}-fold SNR enhancement. In FIG. 2, Nₑ=4. The signals acquired from a single virtual source emission are defined as pₙ for n=1, 2, . . . , Nₛ, where Nₛ is the total number of virtual sources. By simultaneously transmitting ultrasound from Nₛ different virtual sources, the SNR can be enhanced by approximately \sqrt{Nₑ·Nₛ} times compared to single element transmission.

Referring again to FIG. 1, after the series of virtual sources is created, the virtual sources are coded using a coding matrix, as indicated at step 104. Generally, the coding matrix adjusts the amplitude, phase, or both, of the pulses transmitted by a given virtual source. Coding the virtual sources spatially encodes the transmission sequence, which can result in an additional increase in transmit power that provides for an additional increase in the attainable SNR. As one example, the coding matrix can be a Hadamard coding matrix, whose entries are either 1 or −1, representing positive or negative (i.e., inverted) transmission pulses, respectively.

A 2^{k}-th order Hadamard matrix, H₂_{^{k}}, can be constructed using the following construction,

H₂_{^{k}}=H₂⊗H₂_{^{k−1}} for k≥2 (2);

where,

$${H}_{2} = [ \begin{matrix}1 & 1 \\ 1 & - 1\end{matrix} ] ; \qquad ( 3 )$$

and ⊗ denotes the Kronecker product operator. Each row in the Hadamard matrix corresponds to one transmission event, whereas each column corresponds to a different virtual source.

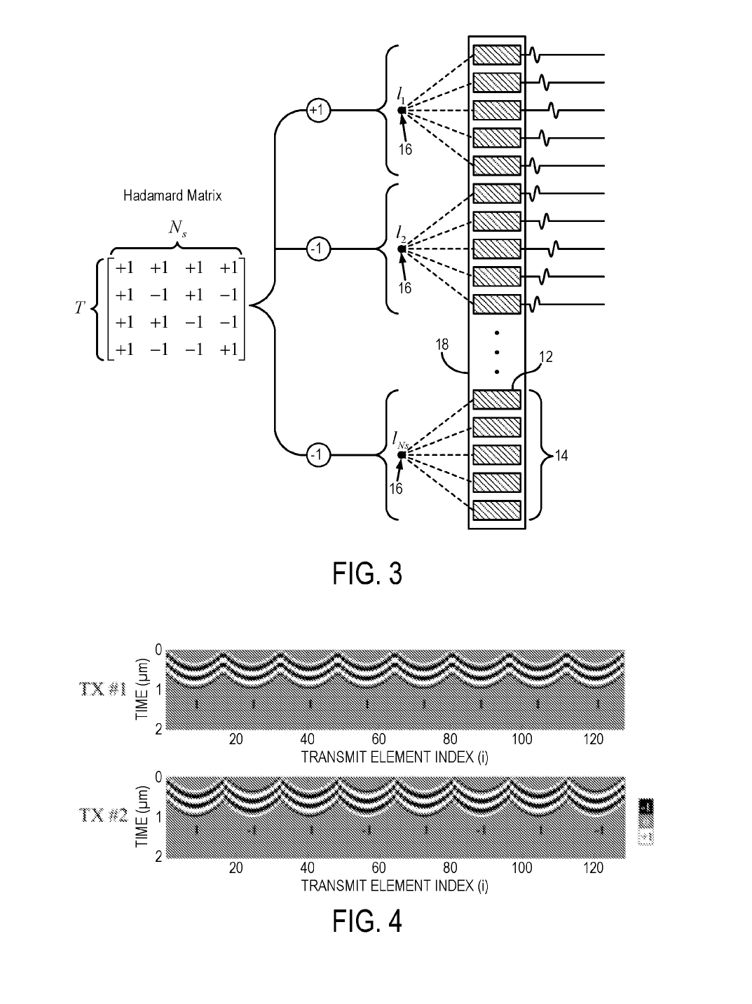

FIG. 3 illustrates an example of a series of coded virtual sources in which a Hadamard coding matrix has been applied to a series of virtual sources 16. In this example, Nₛ=4 virtual sources 16 are created and they each transmit either a positive or a negative (i.e., inverted) pulse according to the corresponding 4th order Hadamard matrix. FIG. 3 illustrates a transmission event utilizing the second row of the Hadamard matrix to code the virtual sources 16.

In the example illustrated in FIG. 3, the ultrasound system excites the transducer array 18 by simultaneously firing the Nₛ virtual sources 16 at lateral locations of l₁, l₂, . . . , l_{N}_{ₛ}. These virtual sources 16 have the same axial depth, l_{z}, in this , example, but different lateral locations, lₓ. Diverging beams are emitted from each sub-aperture 14. Positive and negative (i.e., inverted) pulses are emitted from the sub-apertures 14 with 1 and −1 coding factors, respectively. The echoes received using this configuration of simultaneously activated virtual sources can be defined as mₜ for t=1, 2, . . . , T, where T is the total number of transmission events. Then, the Hadamard encoding process can be described as,

HP=M (4);

where H is the Hadamard coding matrix and P and M are two column vectors,

$$\begin{matrix}P = [ \begin{matrix}{p}_{1} \\ {p}_{2} \\ ⋮ \\ {p}_{Ns}\end{matrix} ] ; & ( 5 ) \\ M = [ \begin{matrix}{m}_{1} \\ {m}_{2} \\ ⋮ \\ {m}_{T}\end{matrix} ] . & ( 6 )\end{matrix}$$

FIG. 4 illustrates wave fronts (2 pulse cycles per wave front) from the first (top row) and second (bottom row) transmission events when using an 8th order Hadamard matrix. In this example, eight virtual sources were created from a 128-element array (i.e., 16 elements per virtual source) and each virtual source transmitted either a positive or a negative pulse according to their lateral and axial locations and the corresponding Hadamard coding factors. The numbers labeled below the wave fronts in FIG. 4 represent the polarities of the transmitted pulses (i.e., “1” stands for a positive pulse whereas “−1” stands for negative, or inverted, pulse).

When Nₑ·Nₛ equals to the total number of transducer elements in the transducer, the amplitude of these two-step encoded signals should be comparable to that in compounding plane (or diverging) wave imaging since all elements are excited in both configurations during one transmission event. The spatial resolution in the USTA method described here, however, can be significantly improved without reducing the frame rate using synthetic transmit focusing. For example, Nₛ, transmission events are used to decode a coding matrix, such as the Hadamard matrix, used to code virtual sources as described above. If Nₛ is equal to the number of tilted angles used in plane/divergent wave compounding, the same ultrafast frame rate can be achieved for both methods.

Referring again to FIG. 1, the transmission schedule defined by the coded virtual sources can be modified to include time intervals between the transmission of each coded virtual source in a single transmission event, as indicated at step 106. In some sequences, the sub-apertures defined by the virtual sources are spatially overlapping. In these situations, the transmission schedule can be modified to include a time interval, or time delay, between the transmission of pulses from virtual sources associated with spatially overlapping sub-apertures.

For instance, the SNR in USTA imaging can be improved by either increasing Nₛ or Nₑ. If the same frame rate is desired, Nₛ can be kept the same while Nₑ is increased to further enhance signal amplitude. This results in spatially overlapping sub-apertures between the virtual sources. When spatially overlapping sub-apertures are implemented, the transmission sequence is adjusted by adding a time interval, Δt, between spatially overlapping sub-apertures. This time interval can be very short, such as on the order of a few microseconds. Ultrasound beams can then be quasi-simultaneously transmitted from the various sub-apertures in a single transmission event.

Another advantage of using spatially overlapping sub-apertures is as follows. To obtain a more uniform beam pattern and less side-lobe effects, apodization can be used to apply less weight on boundary transmitting elements in each of the sub-apertures. However, using apodized sub-apertures, the energy of the transmitted pulse from each virtual source is reduced. To maintain or otherwise increase transmit energy using apodization, spatially overlapping sub-apertures can be used in a single transmission event.

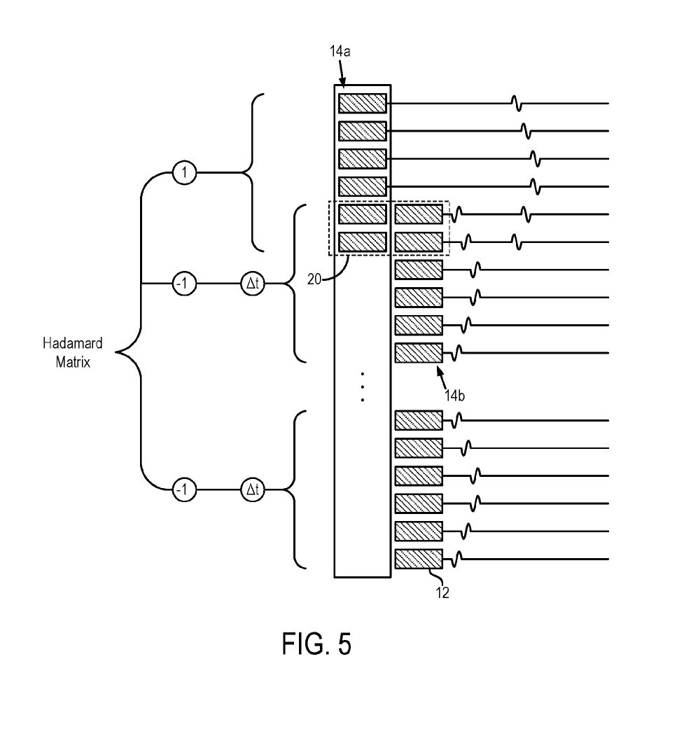

Referring now to FIG. 5, an example of temporally offset spatially overlapping sub-apertures is illustrated. In this example, the fifth and sixth transmitting elements (indicated by dashed box 20) are shared by the first two sub-apertures 14a, 14b. Thus, a time interval, Δt, is added to the second sub-aperture 14b to allow for the repeated emissions of the transducer elements 12 in the spatially overlapping region 20 of the two sub-aperture 14a, 14b. In these configurations, the transducer elements 12 in the spatially overlapping region 20 will emit a longer pulse in each transmission event than the transducer elements 12 that only transmit once because they are not shared by two sub-apertures 14.

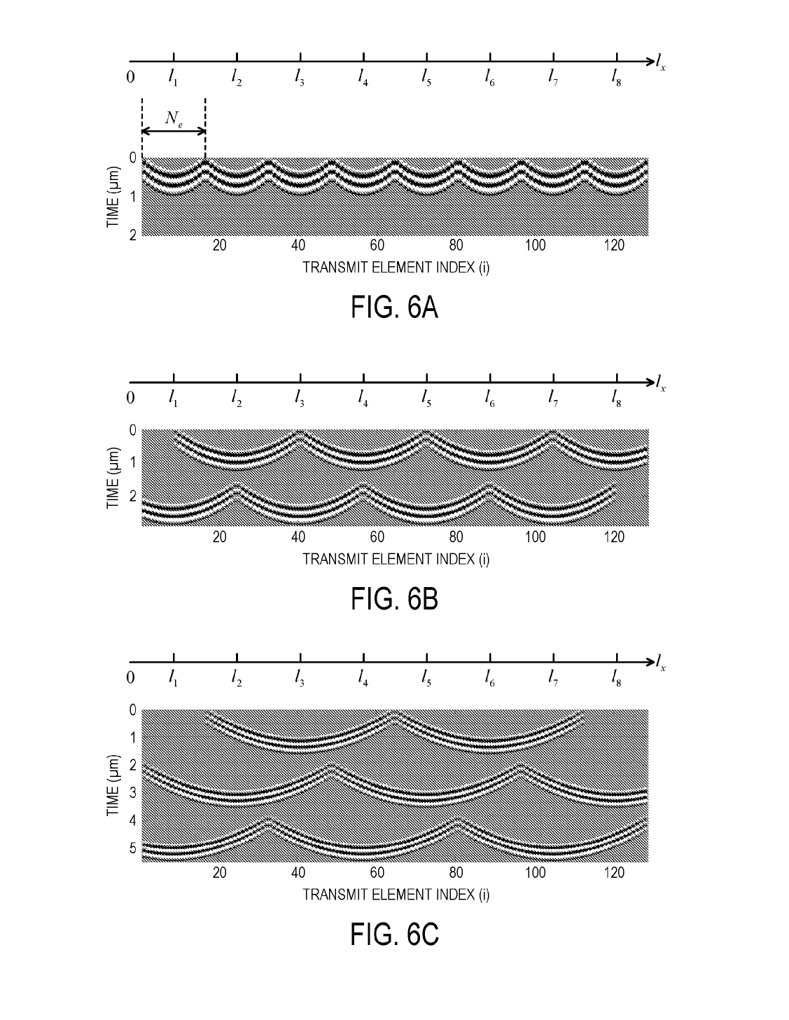

FIGS. 6A-6C illustrate three example pulse designs (2 pulse cycles per wave front) of USTA imaging in one transmission event. In FIG. 6A, 16-element sub-apertures are used, in FIG. 6B, 32-element sub-apertures are used, and in FIG. 6C, 48-element sub-apertures are used, all positive polarities. Eight virtual sources were created for all three configurations with the same f-number (fₙ) and lateral locations lₓ) with focal depths varied to maintain the same f-number. Hence, the same lateral spatial resolutions can be expected for these three example configurations. In addition, the same number of virtual sources (Nₛ) in each example leads to the same frame rate in each example. One advantage of spatially overlapping sub-apertures is that as the size of the sub-aperture (Nₑ) increases, transmit power (and therefore SNR) are gradually increased. One trade-off of increasing the sub-aperture size is a slightly larger dead zone at near field with greater Nₑ due to the longer transmit duration during each transmission event.

After USTA transmission, the received signals (M) undergo decoding steps by multiplying with the inverse of the coding matrix (H⁻¹) used to code the virtual sources to obtain P, which is the equivalent data as obtained when each virtual source is activated individually, but with significantly improved SNR,

P=H⁻¹M (7).

The number of transmission events should be equal to the number of virtual sources (i.e., T=Nₛ) to ensure complete decoding. If T<Nₛ, the decoding is ill-conditioned, and a pseudo-inverse or other regularization method can be used to estimate P. Using Hadamard coding for the encoding pattern has the advantage that the inverse of the Hadamard coding matrix is the Hadamard coding matrix itself multiplied by a constant,

$${H}_{{2}^{k}}^{- 1} = \frac{1}{{2}^{k}} {H}_{{2}^{k}} . \qquad ( 8 )$$

Therefore, the decoding process is stable and can be achieved from simple additions and subtractions, which is convenient for implementation. It will be appreciated by those skilled in the art, however, that other coding matrices can be implemented with decoding being achieved using the inverse of that coding matrix. After decoding, the time shift, Δt, introduced by adding a time interval between transmission of spatially overlapping sub-apertures can be compensated for to realign data from different virtual sources. This compensation can be achieved by shifting the pre-beamformed data axially by the appropriate time determined by Δt.

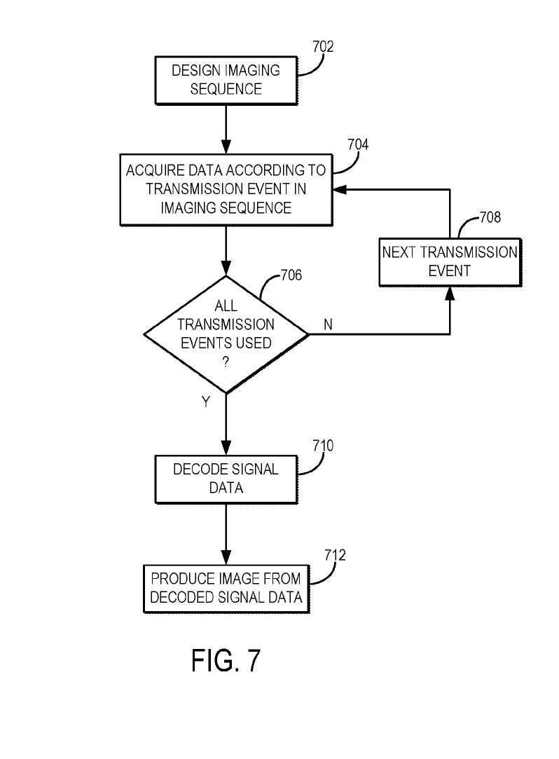

Referring now to FIG. 7, a flowchart is illustrated as setting forth the steps of an example method for imaging a subject using the ultrafast synthetic transmit aperture imaging technique described here. The method includes designing an appropriate imaging sequence for the imaging task at hand, as indicated at step 702. This step can include setting the number of virtual sources, Nₛ, the number of elements in each sub-aperture, Nₑ, the f-number, fₙ, and the locations of each virtual source (lₓ, l_{z}). If any of the sub-apertures are spatially overlapping, this step can also include selecting one or more time intervals, Δt, to be added between transmissions from the spatially overlapping sub-apertures, in a single transmission event. Designing the imaging sequence also includes selecting a coding matrix and applying the coding matrix to the virtual sources. The designed imaging sequence thus defines the position and number of virtual sources, the size and location of the associated sub-apertures, and the timing of how each virtual source should be used to transmit ultrasound in a number of different transmission events.

Using the designed imaging sequence, signal data are acquired from the subject by transmitting ultrasound according to the first transmission event in the designed imaging sequence and receiving signals from the subject in response thereto, as indicated at step 704. A determination is made at decision block 706 whether all of the transmission events in the imaging sequence have been implemented, and if not the next transmission event is selected as indicated at step 708 and used to acquire additional signal data at step 704. When all of the signal data have been acquired, they are decoded as indicated at step 710. For example, the signal data are decoding using an inverse of the coding matrix used to code the virtual sources. An image is then produced from the decoded signal data, as indicated at step 712.

The USTA imaging sequence described here provides improved spatial resolution and SNR compared to standard coherent diverging wave compounding (“DWC”) while still retaining the frame rate. Virtual sources are created and coded by applying a coding matrix (e.g., a Hadamard coding matrix) on corresponding sub-apertures instead single elements. Temporally, multiple ultrasound beams can be emitted separated in time within a single transmission event.

In addition to improving SNR without reducing frame rate, the USTA imaging sequence described here can improve spatial resolution as compared to coherent compounding and multiplane wave imaging. In USTA, the spatial resolution is determined by the f-number of virtual sources (fₙ).

Imaging sequences usually seek best compromise among image quality metrics with acceptable sacrifices. USTA offers both improved resolution and SNR compared to coherent compounding without sacrificing frame rate. The potential high frame rate and improved performance may be useful in ultrafast imaging and related applications such as ultrafast Doppler and shear wave elastography.

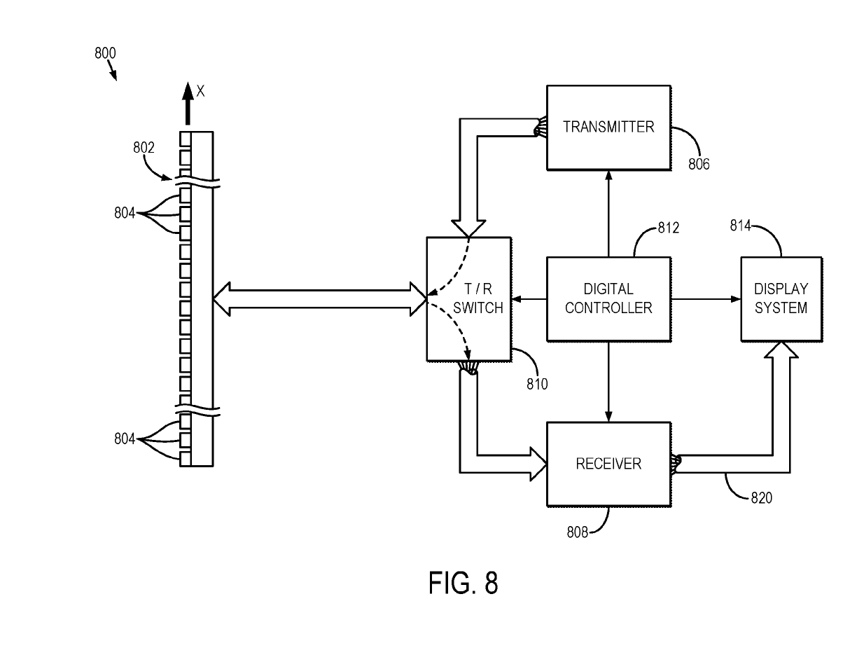

FIG. 8 illustrates an example of an ultrasound system 800 that can implement the ultrafast synthetic transmit aperture imaging techniques described here. The ultrasound system 800 includes a transducer array 802 that includes a plurality of separately driven transducer elements 804. The transducer array 802 can include any suitable ultrasound transducer array, including linear arrays, curved arrays, phased arrays, and so on. When energized by a transmitter 806, each transducer element 802 produces a burst of ultrasonic energy. The ultrasonic energy reflected back to the transducer array 802 from the object or subject under study is converted to an electrical signal by each transducer element 804 and applied separately to a receiver 808 through a set of switches 810. The transmitter 806, receiver 808, and switches 810 are operated under the control of a controller 812, which may include one or more processors. As one example, the controller 812 can include a computer system. 100451 The controller 812 can be programmed to design an imaging sequence using the techniques described above. In some embodiments, the controller 812 receives user inputs defining various factors used in the design of the imaging sequence, which may include the number and location of virtual sources, the f-number for virtual sources, the size of sub-apertures defined by the virtual sources, time intervals to be added between transmissions from spatially overlapping sub-apertures, and so on.

A complete scan is performed by acquiring a series of echo signals in which the switches 810 are set to their transmit position, thereby directing the transmitter 806 to be turned on momentarily to energize each transducer element 804 during a single transmission event according to the designed imaging sequence. The switches 810 are then set to their receive position and the subsequent echo signals produced by each transducer element 804 are measured and applied to the receiver 808. The separate echo signals from each transducer element 804 can be combined in the receiver 808 to produce a single echo signal. As mentioned above, the acquired signals can be decoded using an inverse of a coding matrix used to code the virtual sources used in the imaging sequence. Images produced from the decoded signals can be displayed on a display system 814

The transmitter 806 drives the transducer array 802 according to the imaging sequence such that an ultrasound beam is produced by each sub-aperture according to the coded virtual sources defined in the imaging sequence. If spatially overlapping sub-apertures are used, the transmitter 806 drives the elements 804 in each sub-aperture to transmit an ultrasound beam spaced apart in time by the selected time interval, Δt.

The present disclosure has described one or more preferred embodiments, and it should be appreciated that many equivalents, alternatives, variations, and modifications, aside from those expressly stated, are possible and within the scope of the invention.

1. A method for ultrafast synthetic transmit aperture imaging with an ultrasound system, the steps of the method comprising:
(a) selecting with a computer system, a series of virtual sources that define sub-apertures of ultrasound transducer elements in an ultrasound transducer array;
(b) generating coded virtual sources by applying a coding matrix to the series of virtual sources with the computer system, wherein entries in the coding matrix define a characteristic of transmit signals to be applied to the sub-apertures in each of a plurality of different transmission events;
(c) acquiring coded signal data from a subject by transmitting ultrasound beams to the subject in each of the plurality of transmission events with the sub-apertures in the ultrasound system using the respective coded virtual sources and receiving coded echo signals in response thereto;
(d) decoding the coded signal data with the computer system using an inverse of the coding matrix; and
(e) producing an image of the subject from the decoded signal data using the computer system.

2. The method as recited in claim 1, wherein the coding matrix is a Hadamard coding matrix.

3. The method as recited in claim 1, wherein step (c) includes simultaneously transmitting ultrasound beams from each sub-aperture in each transmission event.

4. The method as recited in claim 1, wherein at least some of the sub-apertures are spatially overlapping sub-apertures and step (c) includes transmitting ultrasound beams with the spatially overlapping sub-apertures in each transmission event spaced apart in time by a time interval.

5. The method as recited in claim 4, wherein step (d) includes processing the coded signal data to temporally realign coded signal data associated with different virtual sources separated in time by the time interval.

6. The method as recited in claim 1, wherein step (a) includes selecting an f-number for each virtual source.

7. The method as recited in claim 1, wherein step (a) includes selecting a lateral location and an axial location for each virtual source.

8. The method as recited in claim 1, wherein step (a) includes selecting a number of transducer elements contained in each sub-aperture.

9. The method as recited in claim 1, wherein step (a) includes selecting a number of virtual sources.

10. The method as recited in claim 1, wherein the characteristic of the transmit signals includes at least one of an amplitude, a phase, or a polarity.

11. The method as recited in claim 1, wherein step (c) includes receiving echo signals with respective sub-apertures used to transmit ultrasound beams that formed the respective echo signals.

12. The method as recited in claim 1, wherein step (c) includes receiving each of the echo signals with all of the sub-apertures.

13. A method for ultrafast synthetic transmit aperture imaging with an ultrasound system, the steps of the method comprising:
(a) selecting with a computer system, a series of virtual sources that define sub-apertures of ultrasound transducer elements in an ultrasound transducer array, wherein at least some of the sub-apertures are spatially overlapping;
(b) generating coded virtual sources by applying a coding matrix to the series of virtual sources with the computer system, wherein entries in the coding matrix define a characteristic of transmit signals to be applied to the sub-apertures in each of a plurality of different transmission events;
(c) acquiring coded signal data from a subject by transmitting ultrasound beams to the subject in each of the plurality of transmission events with the sub-apertures in the ultrasound system using the respective coded virtual sources and receiving coded echo signals in response thereto, wherein the transmitting of ultrasound beams with spatially overlapping sub-apertures is spaced apart in time by a time interval;
(d) decoding the coded signal data with the computer system using an inverse of the coding matrix; and
(e) producing an image of the subject from the decoded signal data using the computer system.

14. The method as recited in claim 13, wherein step (d) includes processing the coded signal data to temporally realign coded signal data associated with different virtual sources separated in time by the time interval.

15. The method as recited in claim 13, wherein the coding matrix is a Hadamard coding matrix.

16. The method as recited in claim 13, wherein step (c) includes simultaneously transmitting ultrasound beams from each sub-aperture in each transmission event.

17. The method as recited in claim 13, wherein step (a) includes selecting at least one of an f-number for each virtual source, a lateral location for each virtual source, an axial location for each virtual source, a number of transducer elements contained in each sub-aperture, or a number of virtual sources.

18. The method as recited in claim 13, wherein the characteristic of the transmit signals includes at least one of an amplitude, a phase, or a polarity.

19. The method as recited in claim 13, wherein step (c) includes receiving echo signals with respective sub-apertures used to transmit ultrasound beams that formed the respective echo signals.

20. The method as recited in claim 13, wherein step (c) includes receiving each of the echo signals with all of the sub-apertures.
